(19) United States
(12) Patent Application Publication
(10) Pub. No.: US 2024/0140902 A1
(43) Pub. Date: May 2, 2024

(54) NOVEL METHOD FOR MANUFACTURING TOMATO LEAFMINER PHEROMONE COMPONENTS

(71) Applicant: MELCHIOR MATERIAL AND LIFE SCIENCE FRANCE, LACQ (FR)

(72) Inventors: Eric GAYON, Monein (FR); Loic GUILLONNEAU, Orleans (FR); Olivier GUERRET, Pern (FR)

(73) Assignee: MELCHIOR MATERIAL AND LIFE SCIENCE FRANCE, LACQ (FR)

(21) Appl. No.: 18/276,763

(22) PCT Filed: Feb. 11, 2022
(86) PCT No.: PCT/FR2022/050251

§ 371 Date: Aug. 10, 2023

(30) Foreign Application Priority Data
Feb. 11, 2021 (FR) FR2101323

(51) Int. Cl. C07C 69/145; A01P 17/00

(52) U.S. Cl. CPC C07C 69/145; A01P 17/00

(57) ABSTRACT

The present invention relates to a method for synthesising a pheromone of general formula selected from the group comprising compounds (I) and (I′), alone or in admixture: (I) (I′) comprising reacting (2E)-pentene-1,5-diyl diacetate with at least one Grignard reagent in the presence of a copper-based catalyst. The method according to the invention is particularly applicable to the selective synthesis of tomato leafminer (Tuta absolute) pheromone comprising: (3E,8Z,11Z)-tetradecatrien-1-yl acetate and (3E,8Z)-tetradecadien-1-yl acetate.

For reasons of public health and management of the agricultural potential of soils, crop treatment technologies against pests are evolving towards more targeted and more environmentally friendly modes of action. As such, the use of sex pheromones to modify the behavior of insects has advantages since these pheromones are specific to each species of pest and are effective, at very low doses, in various types of strategy (trapping, mating disruption for example).

However, a brake on the development of these technologies lies in the cost of access to active molecules. Indeed these molecules often have many possible isomers and selective synthesis technologies are generally expensive.

The tomato leafminer, Tuta absoluta, a tomato pest present on all continents, is a good example of the problems encountered by the person skilled in the art when developing pheromone-based control solutions. Indeed, the main components of the Tuta absoluta pheromone, are:

TABLE 1
main components of the Tuta absoluta pheromone

| | | Proportion in the |
|---|---|---|
| | | pheromone |
| Formula | Name | mixture |
| 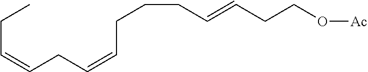 [SMILES: CC/C=C\C/C=C\CCC/C=C/CCOC(C)=O] | (3E,8Z,11Z)- Tetradecatrien-1-yl Acetate | 88 |
| 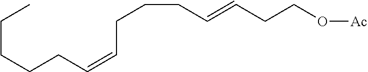 [SMILES: CCCCC/C=C\CCC/C=C/CCOC(C)=O] | (3E,8Z)-Tetradecadien-1-yl Acetate | 12 |

These molecular structures have many positional and configurational isomers. For triene, no less than 8 configurations are possible, but only the 3E,8Z,11Z configuration is active on the insect's olfactory receptors. Some isomers may even have an antagonistic function. It is therefore not only necessary to produce these compounds more economically but also with the highest possible purity. These two requirements are often contradictory and the person skilled in the art has rarely succeeded in reconciling them.

The first known synthesis of the components of the Tuta absoluta pheromone is reported in the publications: A. B. Attygalle & al. Tetrahedron Letters, Vol. 36, No. 31, pp. 5471-5474, 1995 and A. B. Attygalle & al. Bioorganic & Medicinal Chemistry, Vol. 4, No. 3, pp. 305-314, 1996. This academic work describes an interesting synthetic route because it allows to address the problem of selectivity of the synthesis via the reduction of triple bonds into double bonds Z or E. The reaction of construction of the 14 carbon atom structure consists of the reaction of a lithium alkynide with the intermediate IVa:

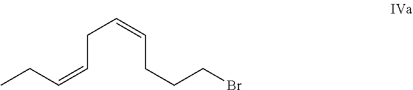

which requires very dilute conditions and therefore high fixed costs. In total it is a yield of 7% which also implies high variable costs. Moreover, the lithiated derivative must be protected by a tetrahydropyran group, which requires an additional deprotection and acetylation step.

On the other hand, the teaching of EP 2639217 consists of a synthetic route analogous to the previous one but which differs because the compound:

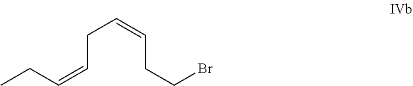

is first transformed into a Grignard reagent then coupled to the compound:

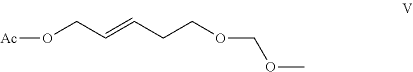

in the presence of a catalyst composed of CuCl₂ and triethylphosphite. The coupling product is then transformed into alcohol then acetylated to produce the desired compound. In this patent, the principle therefore consists in coupling a chain with 9 carbon atoms and 2 unsaturations Z, with a synthon with 5 carbon atoms carrying an unsaturation E. The interest of compound V for the inventors of EP 2639217 consisted of protecting one end of the chain by the methoxymethyl ether function to avoid side reactions during the reaction with the Grignard compound.

However, after the coupling, there are still 2 steps to obtain the target molecule, which greatly reduces the overall yield.

Thus, there remains a need to develop a selective method for obtaining isomerically pure pheromone involving a limited number of reactions and allowing a good yield.

Surprisingly, the applicant has discovered that it was possible to produce a magnesian coupling, in the presence of a catalyst, of the compound II,

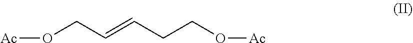 [SMILES: CC(=O)OC/C=C/CCOC(C)=O]

perfectly selective with a substitution of the acetate function in position 1 (allylic position) and no reactivity of the acetate function in position 5 (homoallylic position). Indeed, the coupling between an organomagnesium derivative and a homoallylic acetate derivative (that is to say (3Z-octen-1-yl acetate) catalyzed by a copper(II) complex has been described by Hu and al. Molecules (2012), vol. 17, pp. 12140-12150 and led to the corresponding coupling product with a yield of 87% and a purity of 97%. It was therefore not obvious to the person skilled in the art that the substitution reaction forming the subject of this patent is specific for position 1 (allylic position). The inventors have also observed that the level of substitution products in position 3 is much lower according to the method of the invention than according to the known methods mentioned above. Since the acetate function in position 5 is desired for the structure of the targeted pheromone, the fact that it is not at all affected by the reaction under the conditions of the invention is an advantage of the invention because this allows to avoid protection, deprotection and acetylation reactions which, if they have good yields, nevertheless require significant working time and generate aqueous effluents in large amounts which is detrimental from a sustainable industrial development point of view.

Another advantage noted by the inventors lies in the fact that, when two magnesia R-MgX and R′-MgX are used simultaneously in a given ratio, their reaction products with compound II are then themselves in the same ratio. This allows to prepare pheromone mixtures without having to prepare each component of the mixture individually.

Thus, the present invention relates to a method for synthesizing a pheromone of general formula selected from the group comprising the compounds (I) and (I′), alone or in admixture:

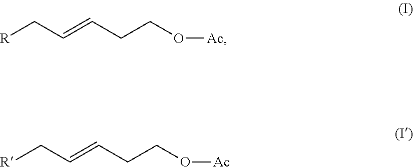

where R is a linear hydrocarbon chain of molecular formula CₙH₂ₙ₋₂ₚ₊₁,

R′ is a linear hydrocarbon chain of molecular formula C_{n′}H_{2n′-2p′+1} with:
n and n′, which are identical or different, are integers comprised between 5 and 17 and
p and p′ corresponding to the number of unsaturations of the hydrocarbon chain, which are identical or different, are integers comprised between 1 and 3,

Ac is an Acetyl radical,

comprising reacting (2E)-pentene-1,5-diyl diacetate of formula (II):

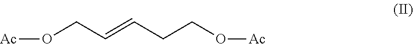 [SMILES: CC(=O)OC/C=C/CCOC(C)=O]

with at least one Grignard reagent selected from the group comprising the compounds (III) and (III′), alone or in admixture:
R-MgX (111), R′-MgX (III′)
   where X is a chlorine or bromine atom;
R and R′ are as defined previously for the compounds of formula (I) and (I′) in the presence of a copper-based catalyst.

Advantageously n and n′, which are identical or different, are comprised between 7 and 15, particularly between 9 and 13.

Also advantageously, p and p′, which are identical or different, are integers comprised between 1 and 3, more particularly between 1 and 2.

p and p′ represent, as indicated, the total number of unsaturations of the chain R or of the chain R′. It is understood that one double bond counts as one unsaturation and that one triple bond counts as two unsaturations. The chain R or R′ may thus comprise one or more double bonds, but also a triple bond associated with a double bond.

In one embodiment, the method is characterized in that the copper-based catalyst further comprises, that is to say is supplemented with, at least one copper ligand selected from the group comprising trialkylphosphine, trialkylphosphite, an amine, and mixtures thereof.

The term “alkyl” in the expression trialkylphosphite or trialkylphosphine corresponds to a C1-C6 alkyl group, in particular methyl, ethyl or propyl.

In one embodiment, the method is characterized in that the ligand is a trialkylphosphite selected from the group comprising triethylphosphite, trimethylphosphite and mixtures thereof.

In one embodiment, the method is characterized in that the reaction is carried out in an aprotic solvent, in particular of the linear or cyclic ether type, in particular selected from the group comprising diethyl ether, methyltertbutyl ether, tetrahydrofuran, methyltetrahydrofuran, tetrahydropyran, methyltetrahydropyran, dioxane and glycol dimethyl ether, and mixtures thereof.

In one embodiment, the method is characterized in that the copper-based catalyst is a copper II salt.

More particularly, the copper II-based catalyst can be selected from the group comprising copper II halides and copper II carboxylates.

In one embodiment, the method is characterized in that the copper II halide is selected from CuCl₂ and CuBr₂; the copper II carboxylate is advantageously selected from copper acetate Cu(OAc)₂ and copper II acetyl acetonate, Cu(Acac)₂.

In the present description, and unless otherwise indicated, when reference is made to equivalent, it is a question of molar equivalent expressed with respect to compound (II) at 1 molar eq.

The invention also relates to a method comprising the following steps:
a. Preparing, in an aprotic solvent, a solution comprising 1 equivalent of (2E)-pentene-1,5-diyl diacetate of formula (II);
b. adding, to the preparation obtained in step a), the copper II-based catalyst at the rate of 0.001 to 0.02 equivalent of copper, the copper possibly being able to be bound optionally to a ligand selected from the group comprising trialkylphosphine, trialkylphosphite, an amine, and mixtures thereof;
c. preparing, in an aprotic solvent identical to or different from the solvent of step a), a solution comprising between 0.95 and 1.25 equivalents of a Grignard reagent selected from the group comprising compounds (III) and (III′), alone or in admixture;
d. adding the solution obtained in step c) to the solution prepared in step b) with stirring and at a reaction temperature below 25° C.;
e. stopping the reaction, in particular by adding 0.1 to 1 equivalent of acetic anhydride to the reaction medium obtained following step d);
f. neutralizing the reaction medium obtained following step e) and extracting the pheromone of formula (I) or (I′) or the mixture of pheromones of formulas (I) and (I′).

According to one embodiment, the method according to the invention is characterized in that the compound (III):compound (III′) molar ratio is comprised between 0:1 and 1:0.

Advantageously, the compound (III):compound (III′) molar ratio is comprised between 0.01:0.99 and 0.99:0.01, particularly between 0.05:0.95 and 0.95:0.05, or even between 0.1:0.9 and 0.9:0.1, or even between 0.2:0.8 and 0.8:0.2, or between 0.3:0.7 and 0.7:0.3, and again between 0.6:0.4 and 0.4:0.6, or even still around 0.5:0.5.

Advantageously, step a) is carried out under an inert atmosphere, for example under nitrogen or argon. This step a) is preferably carried out at a temperature comprised between −20° C. and +60° C., particularly between −10° C. and +10° C.

The amount of copper-based catalyst may advantageously be comprised between 0.005 and 0.03 equivalents of Copper II, or even between 0.01 and 0.02 equivalents.

The amount of Grignard reagent (compound III or compound III′ or compound III+III′) may advantageously be comprised between 1 and 1.2 equivalents, or even between 1 and 1.15 equivalents.

Step c) is also advantageously carried out under an inert atmosphere, for example under nitrogen or argon, preferably at a temperature comprised between −15° C. and 50° C. More preferably, step c) advantageously carried out under an inert atmosphere, for example under nitrogen or argon, is carried out at a temperature comprised between −10° C. and 10° C.

Step d) is carried out with stirring and advantageously at a reaction temperature below 25° C., preferably below 15° C., even more preferably between 8° C. and 12° C. At the end of the addition, the temperature of the medium can advantageously be lowered to 0° C. and the stirring maintained until one of the reagents has disappeared. The disappearance of one of the reagents can be followed by GC (gas chromatography) and/or TLC (thin layer chromatography).

Step e) of stopping the reaction, in particular by adding 0.1 to 1.5 equivalents of acetic anhydride, is advantageously carried out at a temperature maintained below 30° C., preferably at a temperature maintained below or equal to 25° C. Indeed the addition of acetic anhydride is exothermic and it is preferable to maintain the temperature below 30° C.

The neutralization step f) is advantageously carried out using an ammonium chloride solution at a concentration and in an amount suitable for adjusting the pH to a value of the order of 7 to 8, without excessively diluting the medium. The ammonium chloride solution can be a solution at a concentration of 10 to 20% w/w, or even about 16% w/w. The pH will advantageously be adjusted to a value comprised between 7 and 8, particularly between 6.5 and 8.

The invention also relates to a composition comprising a pheromone of general formula selected from the group comprising compounds (I) and (I′), alone or in admixture:

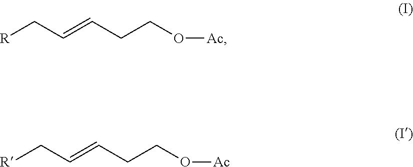

where:
R is a linear hydrocarbon chain of molecular formula CₙH₂ₙ₋₂ₚ₊₁,
R′ is a linear hydrocarbon chain of molecular formula C_{n′}H_{2n′-2p′+1} with:
n and n′, which are identical or different, are integers comprised between 5 and 17;
p and p′ corresponding to the number of unsaturations in the hydrocarbon chain, which are identical or different, are integers comprised between 1 and 3;
Ac is an acetyl radical, and containing, in % by weight of the composition, less than 2%, preferably less than 1.9%, of a compound selected from the group comprising compounds (VI) and (VI′), alone or in admixture:

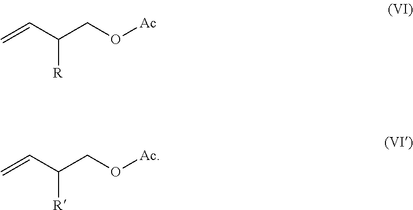

Advantageously, the amount of compound selected from the group comprising compounds (VI) and (VI′), alone or in admixture, is less than 1.8%, or even less than 1.7%, less than 1.6%, less than 1.5%, in % by weight of the composition.

In particular R and R′ are selected from the group (3Z,6Z)-nonadien-1-yl and the group (3Z)-nonen-1-yl.

In particular, the method is applicable to the selective synthesis of tomato leafminer (Tuta absoluta) pheromone comprising: (3E,8Z,11Z)-tetradecadien-1-yl acetate on the one hand and (3E,8Z)-tetradecadien-1-yl acetate, alone or in admixtures.

Thus an advantage of the method according to the invention lies in the selectivity of the reaction with respect to substitutions in position 3. Indeed, the coupling reaction between the compound (II) and the Grignard reagent(s) (Ill or III′ or a mixture III/III′) in the presence of a copper II-based catalyst, in particular a copper II salt, allows to promote the products (I) and/or (I′):

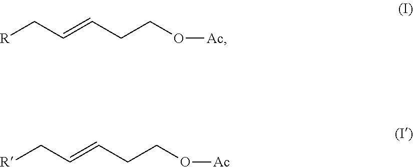

corresponding to an allylic substitution in position 1 while limiting the products (VI) and (VI′):

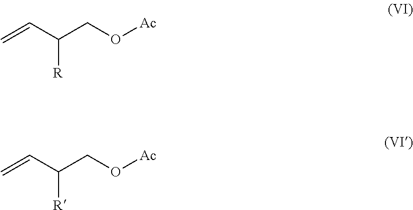

corresponding to an allylic substitution in position 3.

The products (VI) and/or (VI′) represent particularly harmful impurities because they are not part of the family of pheromones with fatty chains of Lepidoptera, which induces problems of toxicity and eco-toxicity.

Reducing such impurities obviously has an economic advantage but above all, it allows to obtain a more effective pheromone because the impurities can have anti-synergistic roles with the pheromones, the insect being able to be repelled by an impurity even in small amounts as reported in the publication Kawazu & al. Journal of Chemical Ecology (2007), vol. 33 pp. 1978-1985.

The inventors have found that the rate of substitution products in position 3 is much lower according to the method of the invention than according to the known methods mentioned above and that this contributes to a greater effectiveness of the products for combating insects.

The method according to the invention is particularly advantageous because the products (VI) and/or (VI′) corresponding to an allylic substitution in position 3 are particularly difficult to separate from the product(s) corresponding to an allylic substitution in position 1, by methods known to the person skilled in the art because their boiling points are very close. It was therefore essential to minimize the content of substitution product(s) in position 3 before starting the purification steps. This is what the method according to the invention allows.

Another advantage of the present method compared to the method taught by document EP 2639217 is also that the use of compound (II) allows to obtain the desired molecules without the need for subsequent protection, deprotection and acetylation reactions taught by EP2639217. This allows a real gain in the number of reactions involved and therefore in yield; which makes it a much more economically advantageous method.

The method according to the invention is detailed below.
It begins with a first step in which , on the one hand, a solution of one equivalent of (2^{E})-pentene-1,5-diyl diacetate II is prepared in particular under an atmosphere of an inert gas such as nitrogen or argon, for example at a temperature comprised between −15° C. and 50° C., preferably between −10° and 10° C.:

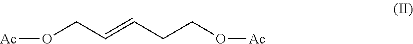 [SMILES: CC(=O)OC/C=C/CCOC(C)=O]

in an aprotic solvent, in particular of the ether type, more particularly diethyl ether, methyltertbutylether, tetrahydrofuran, methyltetrahydrofuran, tetrahydropyran, methyltetrahydropyran, dioxane or else glycol dimethyl ether, then the copper II-based catalyst is introduced into this mixture, optionally in the presence of a ligand, characterized in that it comprises between 0.001 and 0.02 equivalents of copper II salt such as a copper II halide (such as CuCl₂, CuBr₂) or a copper carboxylate (such as copper acetate Cu(OAc)₂ or copper II acetyl acetonate Cu(Acac)₂).
In a second step, always advantageously under an inert atmosphere of nitrogen or argon, a solution is prepared separately in an aprotic solvent of the ether type, particularly diethylether, methyltertbutylether, tetrahydrofuran, methyltetrahydrofuran, tetrahydropyran, methyltetrahydropyran, dioxane or else glycol dimethyl ether, which may be identical to or different from the solvent selected in the first step, containing between 0.95 and 1.25 equivalents of a Grignard reagent selected from the group comprising compounds (III) and (III′), alone or in admixture:
R-MgX (III), R′-MgX (III′)
where X is a chlorine or bromine atom,
R is a linear hydrocarbon chain of molecular formula CₙH₂ₙ₋₂ₚ₊₁,
R′ is a linear hydrocarbon chain of molecular formula C_{n′}H_{2n′-2′p+1} with:
n and n′, which are identical or different, are integers comprised between 5 and 17 and
p and p′ corresponding to the number of unsaturations of the hydrocarbon chain, which are identical or different, are integers comprised between 1 and 3:
Ac is an acetyl radical.
Preferably, the amount of Grignard reagent (compound III or compound III′ or mixture of compound III+III′) is comprised between 1.05 and 1.15 equivalents. This solution is brought to a temperature comprised between −15° C. and 50° C., preferably between −10° C. and 10° C.
In a third step, the solution obtained in the second step is added to the solution prepared in step 1 while maintaining vigorous stirring and a reaction temperature below 25° C., preferably below 15° C., even more preferably between 8 and 12° C. At the end of the addition, the temperature of the medium is lowered to 0° C. and stirring is maintained until one of the reagents disappears.
In a fourth step, the reaction is stopped by adding between 0.1 and 1.5 equivalents of acetic anhydride while maintaining a temperature below 30° C., preferably at a temperature below or equal to 25° C.
In a fifth step, the medium is neutralized by means of an ammonium chloride solution, preferably taking care not to lower the pH below 7, or even not below 6.5. Then the product is extracted several times using an organic solvent selected by a person skilled in the art. The organic phases are then dried, concentrated then distilled to isolate the final product of general formula selected from the group comprising the compounds (I) and (I′), alone or in admixture:

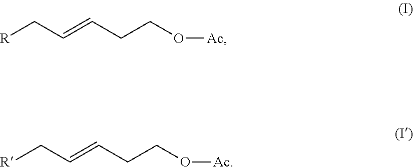

The applicant has thus further found that it was possible to use, as reagent prepared in step 2, a Grignard reagent comprising a single product such as product (III) or product (III′) but also a mixture of products (III+III′), in a selected ratio. The final product is thus a product (I) or a product (I′), or else a mixture of the two products:

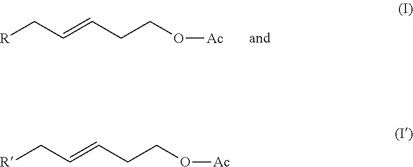

in a ratio equivalent to that of their Grignard reactive precursor products. Thus if the only product (III) is used, the only product (I) will be obtained; if the only product (III′) is used the only product (I′) will be obtained and if a mixture (III+III′) is used in a given molar ratio III:III′, a mixture of products (I+I′) will be obtained in an equivalent molar ratio I:I′. This discovery allows to simplify the routes of synthesis of pheromonel mixtures, particularly at the industrial level since there is no longer any need to mobilize reaction capacities for one of the components, then for the second and finally for the mixture: the mixture is directly obtained in the desired proportions at the end of the synthesis.

Another object of the invention is to obtain a composition comprising the product of formula,

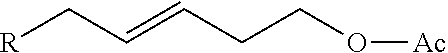

characterized in that the content of impurity of formula

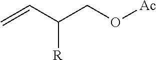

does not exceed 2%, in % by weight, and preferably less than 1.9%, or even less than or equal to 1.6% in particular when the compound R is the group (3Z,6Z)-nonadien-1-yl or the group (3Z)-nonen-1-yl.

The present invention finally relates to the use of a composition according to the invention, comprising a pheromone of general formula selected from the group comprising the compounds (I) and (I′), alone or in admixture as further explained for the protection of crops against the Tuta absoluta pest.

Finally, the invention also relates to a method for protecting an agricultural plot against the Tuta absoluta pest comprising the application within said plot, of a composition according to the invention comprising a pheromone of general formula selected from the group comprising compounds (I) and (I′), alone or in a mixture as set out above.

### EXAMPLES

Raw materials and solvents are commercially available from Sigma Aldrich. Compound II is either purchased from a supplier or manufactured according to the publication S. Olsen & al. Acta Chimica Scandinavica 6 (1952) pp 641-645. Compound IVb is prepared according to patent EP 2639217.

The analytical method consists of an analysis by gas chromatography (GC) on an HP 5890 Series II apparatus equipped with an FID detector. The chromatographic column is an Innowax 30 m, 0.25 mm, 0.25 μm column, the vector gas being helium.

The oven follows the following temperature profile: T0=150° C., Initial time 10 min. Gradient 20° /min, Final temperature: 200° C. Duration 7 min.

The injector is at 250° C., the detector at 300° C.

The injected volume is 1 μl. The concentration of the sample is 4 mg/l in ethyl acetate (AcOEt).

### Example Outside the Invention: synthesis of the Pheromone Mixture (3E,8Z,11Z)-tetradecatrien-1-yl acetate-(3E,8Z)-tetradecadien-1 -yl acetate According to the Conditions Reported in Patent Application EP 2 639 217 A1

A. Synthesis of (3E,8Z,11Z)-tetradecatrien-1-yl methoxymethyl ether

Magnesium (3.43 g, 141 mmol) and THF (59 mL, 52 g) are successively introduced into a reactor, then the medium is stirred at 62° C.±2° C. for 30 min. The (3Z,6Z)-nonadien-1-yl chloride (2.1 g, 134 mmol) is then added dropwise, ensuring that the temperature of the reaction medium is comprised between 60° C. and 65° C. The reaction medium is then stirred at 72° C.±2° C. for 2 hours in order to generate (3Z,6Z)-nonadien-1-yl-magnesium chloride.

In another reactor, copper (II) chloride (0.074 g, 0.548 mmol), triethylphosphite (0.79 mL, 4.62 mmol) and THF (92 mL, 82 g) are successively introduced. 5-acetoxy-(E3)-3-pentenylmethoxymethyl ether (22.91 g, 122 mmol) in solution in THF (92 mL, 82 g) is then added between 5° C. and 10° C. then the reaction medium is then stirred between 0° C. and 5° C. for 30 min. Next, the previously prepared solution of (3Z,6Z)-nonadien-1-yl-magnesium chloride is introduced dropwise, ensuring that the temperature of the reaction medium is comprised between 0 and 5° C. At the end of the introduction, the reaction medium is stirred between 5° C. and 10° C. for 40 min then neutralized with ammonium chloride (1.46 g) then with a 20% w/w hydrochloric acid solution (38 g). The aqueous phase is discarded, the organic phase is concentrated under reduced pressure then the residue obtained is distilled under reduced pressure to give (3E,8Z,11Z)-tetradecatrien-1-yl methoxymethyl ether (24.2 g; yield=78%) which contains 2.7% impurity resulting from the substitution in position 3.

B. Synthesis of (3E, 8Z,11 Z)-tetradecatrien-1-yl acetate

(3E,8Z,11Z)-tetradecatrien-1-yl methoxymethyl ether (24.2 g, 95.8 mmol) and methanol (61.8 g) are placed in a reactor equipped with a distillation column and the reaction medium is stirred at 45° C.±2° C. A 20% w/w hydrochloric acid solution (35.8 g) is then added drop by drop over 1 hour, ensuring that the temperature is comprised between 45° C. and 50° C. At the end of the introduction, the reaction medium is then heated to 55° C. and stirred for 1 hour. The reaction by-products (dimethoxymethane, methanol) are then eliminated by distillation while being placed under a vacuum of 450 mmHg then the residue obtained is stirred for 9 hours. After stirring, the medium is cooled to 25° C. and extracted with hexane (60 g). The organic phase is washed with brine then with an aqueous solution of sodium bicarbonate. The organic phase is then concentrated under reduced pressure then the residue obtained is distilled under reduced pressure to give (3E,8Z,11Z)-tetradecatrien-1-ol (18 g; yield=90%). The (3E,8Z,11Z)-tetradecatrien-1-ol obtained (18 g, 69.2 mmol), toluene (41.2 g), acetic anhydride (1.76 g, 17.2 mmol) and dimethylaminopyridine (0.176 g) are successively introduced into a reactor then the mixture is heated to around 55° C. Acetic anhydride (7.07 g, 69.2 mmol) is once again introduced dropwise between 65° C. and 70° C. over 30 minutes then the reaction medium is stirred at 70° C. for 1 hour. The reaction medium is then cooled to 30° C. then the reaction is quenched by adding water (27.5 g). The two phases obtained are decanted then the organic phase is washed with brine then with an aqueous solution of sodium bicarbonate. The organic phase is then concentrated under reduced pressure then the residue obtained is distilled under reduced pressure to give (3E,8Z,11Z)-tetradecatrien-1-yl acetate (17 g; yield=98%) which contains 2.7% impurity resulting from substitution in position 3.

C. Synthesis of (3E,8Z)-tetradecadien-1-yl methoxymethyl ether

Magnesium (1.56 g, 64.0 mmol) and THF (6.7 mL, 6 g) are successively introduced into a reactor, then the medium is stirred at 62° C.±2° C. for 30 min. The (3Z)-nonen-1-yl chloride (8.87 g, 55.2 mmol) is then added dropwise, ensuring that the temperature of the reaction medium is comprised between 60° C. and 65° C. The reaction medium is then stirred at 72° C.±2° C. for 2 hours in order to generate (3Z)-nonen-1-yl-magnesium chloride.

In another reactor, copper (II) chloride (0.033 g, 0.248 mmol), triethylphosphite (0.36 mL, 2.1 mmol) and THF (4 mL, 3.7 g) are introduced successively. 5-acetoxy-(E3)-3-pentenylmethoxymethyl ether (10.40 g, 55.2 mmol) in solution in THF (4 mL, 3.7 g) is then added between 5° C. and 10° C. then the reaction medium is subsequently stirred between 0° C. and 5° C. for 30 min. Subsequently, the previously prepared solution of (3Z)-nonen-1-yl-magnesium chloride is introduced dropwise, ensuring that the temperature of the reaction medium is comprised between 0 and 5° C. At the end of the introduction, the reaction medium is stirred between 5° C. and 10° C. for 40 min then quenched with ammonium chloride (0.66 g) then with a 20% w/w hydrochloric acid solution (17 g). The aqueous phase is discarded, the organic phase is concentrated under reduced pressure then the residue obtained is distilled under reduced pressure to give (3E,8Z)-tetradecadien-1-yl methoxymethyl ether (25 g; yield=80%) which contains 2.5% impurity resulting from the substitution in position 3.

D. Synthesis of (3E, 8Z)-tetradecadien-1-yl acetate

(3E,8Z)-tetradecadien-1-yl methoxymethyl ether (25 g, 98.3 mmol) and methanol (63.4 g) are placed in a reactor equipped with a distillation column and the reaction medium is stirred at 45° C.±2° C. A 20% w/w hydrochloric acid solution (36.7 g) is then added dropwise over 1 hour, ensuring that the temperature is comprised between 45° C. and 50° C. At the end of the introduction, the reaction medium is then heated to 55° C. and stirred for 1 hour. The reaction by-products (dimethoxymethane, methanol) are then eliminated by distillation while being placed under a vacuum of 450 mmHg then the residue obtained is stirred for 9 hours. After stirring, the medium is cooled to 25° C. and extracted with hexane (62 g). The organic phase is washed with brine then with an aqueous solution of sodium bicarbonate. The organic phase is then concentrated under reduced pressure then the residue obtained is distilled under reduced pressure to give (3E,8Z)-tetradecadien-1-ol (17 g; yield=82%).

The (3E,8Z)-tetradecadien-1-ol obtained (16 g, 76 mmol), toluene (45 g), acetic anhydride (1.93 g, 18.9 mmol) and dimethylaminopyridine (0.193 g) are successively introduced in a reactor then the mixture is heated to around 55° C. Acetic anhydride (7.77 g, 76 mmol) is again introduced dropwise between 65° C. and 70° C. over 30 minutes then the reaction medium is stirred at 70° C. for 1 hour. The reaction medium is then cooled to 30° C. then the reaction is quenched by adding water (30 g). The two phases obtained are decanted then the organic phase is washed with brine then with an aqueous solution of sodium bicarbonate. The organic phase is then concentrated under reduced pressure then the residue obtained is distilled under reduced pressure to give (3E,8Z)-tetradecadien-1-yl acetate (18 g; yield =94%) which contains 2.5% of impurity resulting from the substitution in position 3.

E. Preparation of the Pheromone Mixture (3E,8Z,11Z)-tetradecatrien-1-yl-acetate-(3E,8Z)-tetradecadien-1-yl acetate from Pheromones Previously Synthesized According to the Conditions Reported in Patent Application EP 2 639 217 A1

17.0 g of (3E,8Z,11Z)-tetradecatrien-1-yl acetate obtained in example B and 2.32 g of (3E,8Z)-tetradecadien-1-yl acetate obtained in example D are mixed then the pheromone obtained is analyzed by CPG.

GC analysis: tr=20.4 min ((3E,8Z)-tetradecadien-1-yl acetate, 9.5%), tr=20.8 min ((3E,8Z,11Z)-tetradecadien-1-yl acetate, 76.2%) that is to say in total a content of 85.7% of the pheromone mixture. Content of substitution impurities in position 3: 2.5%.

### Example 1: Synthesis of (3E,8Z,11Z)-tetradecatrien-1-yl acetate

Tetrahydrofuran (9.5 mL) then magnesium (0.47 g; 18.79 mmol) are introduced into a 50 mL three-necked flask, under an inert atmosphere and magnetic stirring. Dibromoethane (0.1 mL; 1.25 mmol) is added and the reaction medium is brought to a temperature of 65° C. After maintaining this same temperature for 45 minutes, (3Z,6Z)-nonadien-1-yl chloride (2.84 g; 1.34 mmol) is added dropwise. The mixture thus obtained is stirred at reflux for 2 hours to generate (3Z,6Z)-nonadien-1-yl magnesium chloride.

In a second 50 mL three-necked flask, under an inert atmosphere and magnetic stirring, a solution of copper (II) chloride (9.6 mg; 0.0715 mmol; 0.004 eq.) and triethylphosphite (101 mg; 0.0462 mol; 0.034 eq) in tetrahydrofuran (1.3 mL) is prepared then cooled to 0° C. A solution of (2E)-pentene-1,5-diyl diacetate (2.8 g; 15.04 mmol) in tetrahydrofuran (1.3 mL) is then introduced.

After 30 minutes of stirring at 0° C., the (3Z,6Z)-nonadien-1-yl magnesium chloride solution previously prepared is added dropwise to the above mixture. At the end of the introduction, the reaction medium is maintained for one hour at 0° C. then for 16 hours at room temperature.

The reaction medium is neutralized by adding a saturated aqueous solution of ammonium chloride (20 mL). The aqueous phase is then extracted with methyl tert-butyl ether (3×20 mL), dried on MgSO₄, filtered and concentrated under reduced pressure to yield a crude (m=3.6 g) which is purified by column chromatography on silica gel (Eluant: Heptane/AcOEt: 90/10) to give (3E,8Z,11Z)-tetradecatrien-1-yl acetate (2.0 g; 53%).

GC analysis: tr=20.8 min Purity: 94%—content of substitution impurity in position 3: 0.93%.

¹H NMR analysis (CDCl₃): compliant.

No impurity corresponding to a substitution in position 5 of the (2E)-pentene-1,5-diyl diacetate is detected, which highlights the advantage of the invention, which avoids a protection step, another one of deprotection then another one of acetylation which, even if they can have high yields, consume other raw materials (here methanol, dimethylaminopyridine, toluene, acetic acid and sodium chloride) generate significant amounts of aqueous effluents and mobilize resources over longer periods of time.

### Example 2: Synthesis of (3E,8Z)-tetradecadien-1-yl acetate

Tetrahydrofuran (9.5 mL) then magnesium (0.45 g; 18.79 mmol) are introduced into a 50 mL three-necked flask, under an inert atmosphere and magnetic stirring.

Dibromoethane (0.1 mL); 1.25 mmol) is added and the reaction medium is brought to a temperature of 65° C. After maintaining 45 minutes at this same temperature, the (3Z)-1-chlorononene (2.84 g; 1.34 mmol) is added dropwise. The mixture thus obtained is stirred at reflux for 2 hours to generate (3Z)-nonen-1-yl magnesium chloride.

A solution of copper (II) chloride (9.6 mg; 0.0715 mmol; 0.004 eq.) and triethylphosphite (101 mg; 0.0462 mol; 0.034 mmol) in tetrahydrofuran (1.3 mL) is prepared then cooled to 0° C. in a second 50 mL three-necked flask, under an inert atmosphere and magnetic stirring. A solution of (2E)-pentene-1,5-diyldiacetate (2.8 g; 15.04 mmol) in tetrahydrofuran (1.5 mL) is then introduced. After stirring for 30 minutes at 0° C., the previously prepared Grignard solution is added dropwise over 35 minutes. At the end of the introduction, the reaction medium is maintained for 1 hour at 0° C. then for 16 hours at ambient temperature.

The reaction medium is neutralized by adding a saturated aqueous solution of ammonium chloride (25 mL). The aqueous phase is then extracted with methyl tert-butyl ether (3×20 mL), dried on MgSO₄, filtered and concentrated under reduced pressure to yield a crude (m=3.7 g) which is purified by chromatography on silica gel (Eluent: Heptane/AcOEt: 90/10) to give (3E,8Z)-tetradecadien-1-yl acetate (2.5 g; 69%).

GC analysis: tr=20.4 min Purity: 97.7%—content of substitution impurity in position 3: not detected.

¹H NMR analysis (CDCI3): compliant.

No impurity corresponding to a substitution in position 5 of the (2E)-pentene-1,5-diyl diacetate is detected in this example, which highlights the advantage of the invention, which avoids a protection step, another one of deprotection then another one of acetylation which, even if they can have high yields, consume other raw materials, generate significant amounts of aqueous effluents and mobilize resources over longer periods of time.

### Example 3: Preparation of the Pheromone Mixture from Examples 1 and 2

0.95 g of the compound obtained in Example 1 and 0.1 g of the compound obtained in Example 2 are mixed in a flask under nitrogen.

GC analysis: tr=20.4 min ((3E,8Z)-tetradecadien-1-yl acetate, 8.1%), tr=20.8 min ((3E,8Z,11Z)-tetradecatrien-1-yl acetate, 86%). Content of substitution impurities in position 3: 0.85%.

If these examples are compared with example E outside the invention, it will be found that the content of substitution impurities in position 3 is much lower and that the purity of the pheromone mixture is 86%+8.1%=94.1% against only 85% in example E outside the invention.

### Example 4: Synthesis of the Pheromone Mixture (3E,8Z,11Z)-tetradecatrien-1-yl-acetate-(3E,8Z)-tetradecadien-1-yl acetate

Tetrahydrofuran (94 mL) then the magnesium (4.57 g; 188 mmol) are introduced in a 250 mL three-necked flask, under an inert atmosphere and magnetic stirring. Dibromoethane (1.1 mL; 12.5 mmol) is added then the reaction medium is brought to a temperature of 65° C. After maintaining 30 minutes at this same temperature, a mixture of (3Z,6Z)-nonadien-1-yl chloride (25.5 g; 160.7 mmol) and (3Z)-1-chlorononene (2.9 g; 18.0 mmol) is added dropwise. The solution thus obtained is stirred at reflux for 2 hours to generate the Grignard reagent.

A solution of copper (II) chloride (94 mg; 0.72 mmol; 0.004 eq.) and triethylphosphite (1.01 g; 6.08 mmol; 0.034 eq) in tetrahydrofuran (13 mL) is prepared then cooled to 0° C. in a second 250 mL three-necked flask, under an inert atmosphere and magnetic stirring. A solution of (2E)-pentene-1,5-diyl diacetate (25.0 g; 134 mmol) in tetrahydrofuran (1.3 mL) is then introduced.

After stirring for 30 minutes at 0° C., the previously prepared Grignard solution is added dropwise to the above mixture over 90 minutes. At the end of the introduction, the reaction medium is maintained for one hour at 0° C. then for 16 hours at room temperature.

The reaction medium is neutralized by adding a saturated aqueous solution of ammonium chloride (150 mL). The aqueous phase is then extracted with methyl tert-butyl ether (2×100 mL), dried on MgSO₄, filtered and concentrated under reduced pressure to yield a crude (m=38.2 g) which is purified by distillation under reduced pressure (1.1 mmHg; Bath temp=150 to 160° C.; Head temp=110 to 120° C.). Two fractions of the (3E,8Z,11Z)-tetradecatrien-1-yl acetate/(3E,8Z)-tetradecadien-1-yl acetate mixture were obtained and combined after NMR analysis.

GC analysis: tr=20.4 min (((3E,8Z)-tetradecadien-1-yl acetate, 10.8%) tr=20.8 min ((3E,8Z,11Z)-tetradecatrien-1-yl acetate 80.5%) that is to say a total content of 91% of the pheromonal mixture. Content of substitution impurities in position 3: 1.12%.

### Example 5: Synthesis of the Pheromone Mixture (3E,8Z,11Z)-tetradecatrien-1-yl acetate—(3E,8Z)-tetradecadien-1-yl acetate on a Scale of 1 kg with Stopping of the Reaction with Acetic Anhydride

Tetrahydrofuran (3.36 kg) then the magnesium (150 g; 6.25 mol) are introduced in a 5 L reactor, under an inert atmosphere and mechanical stirring. Dibromoethane (41 g; 0.39 mol) is added then the reaction medium is brought to a temperature of 65° C. After maintaining 30 minutes at this same temperature, a mixture of (3Z,6Z)-nonadien-1-yl chloride (825 g; 5.2 mol) and (3Z)-1-chlorononene (116 g; 0.7 mol) is added slowly. The solution thus obtained is stirred at reflux for 2 hours to generate the Grignard reagent then brought back to 10° C.

A solution of copper (II) chloride (2.9 g; 0.022 mol; 0.004 eq.) and triethylphosphite (30.6 g; 0.184 mol; 0.034 eq.) in tetrahydrofuran (0.409 kg) is prepared then cooled to 0° C. in a second 20 L reactor, under an inert atmosphere and mechanical stirring. A solution of (2E)-pentene-1,5-diyldiacetate (1 kg; 5.3 mol) in tetrahydrofuran (0.478 kg) is then introduced.

After stirring for 30 minutes at 0° C., the previously prepared Grignard solution is added to the previous mixture over 70 minutes. At the end of the introduction, the reaction medium is maintained for one hour at 0° C. then for 16 hours at room temperature. After checking by chromatography that the reagents have disappeared, 82.2 g of acetic anhydride are added without exceeding 25° C.

The reaction medium is neutralized by adding an aqueous solution of ammonium chloride (3.8 kg at 16% by weight). The aqueous phase is then extracted with methyl tert-butyl ether (0.8 kg), dried on MgSO₄, filtered and concentrated under reduced pressure to yield a crude (m=38.2 g) which is purified by distillation under reduced pressure (1.1 mmHg; Bath temp=150 to 160° C.; Head temp=110 to 120° C.). Two fractions of the mixture (3E,8Z,11Z)-tetradecatrien-1-yl acetate/(3E,8Z)-tetradecadien-1-yl acetate were obtained and combined after NMR analysis (1.049 kg).

GC analysis: tr=20.4 min (11.7%), tr=20.8 min (81.96%) that is to say a total content of 93.5% of the pheromonal mixture. Content of substitution impurities in position 3: 1.58%.

This example illustrates the interest of the invention which allows to directly synthesize the pheromone mixture with the correct proportions of the two components by reacting the diacetate of (2E)-pentene-1,5-diyl directly on the mixture of the two magnesium precursors in the proportions of the final mixture. This avoids having to synthesize 2 compounds then having to mix them. From an industrial point of view, this reduces the downtime of resources, which illustrates the industrial interest of the method according to the invention.

### Example 6: Comparative Trapping Test with a Pheromone According to the Invention and Outside the Invention

### Preparation of Plastic Diffusers

0.1 g of pheromonel mixture obtained according to example 3 (Solution S1) or according to example E outside the invention (Solution S0) is diluted in 10 ml of pentane.

3 ml of solution S0 or S1 are placed in the flask of a 200 ml rotary evaporator in the presence of 15 septa of the Precision Seal® rubber septa 7 mm type, rotated at room temperature and atmospheric pressure for one hour at the speed of 120 rpm. 15 diffusers each loaded on average with 2 mg of pheromone mixture according to the invention or outside the invention are thus obtained.

### Implementing a Comparison Test:

10 pheromone traps of the red Delta type, each equipped with a sticky plate are placed in a greenhouse of 1 ha. A septum loaded with pheromone according to the invention (population of traps P1) is placed on 5 of them and the same is done with the septa loaded with pheromone outside the invention (population of traps P0) on the 5 others. The traps are checked every week, that is to say the Tuta absoluta moths trapped on the sticky plate are counted and the sticky plate is replaced with a new one. The septa are renewed after 4 weeks. Moreover, in order to limit the effects of local insect concentration, the traps are rotated every week.

### Results:

TABLE 1
number of moths trapped per family
of 5 traps (cumulative) per week

| weeks | 1 | 2 | 3 | 4 | 5 | 6 | 7 | 8 | 9 | 10 | 11 | 12 | Total |
|---|---|---|---|---|---|---|---|---|---|---|---|---|---|
| P0 | 1 | 0 | 5 | 4 | 1 | 33 | 58 | 74 | 33 | 1 | 0 | 1 | 211 |
| P1 | 0 | 3 | 12 | 8 | 0 | 40 | 72 | 81 | 45 | 8 | 0 | 0 | 269 |

Better capture of the moths is thus observed with the pheromone containing the least impurities resulting from the substitution in position 3, namely the pheromone synthesized according to the method according to the invention. This demonstrates the negative role of these impurities on the attractiveness of pheromonel mixtures used as baits.

### Example 7: Comparative Tests in Greenhouses for the Treatment of Tomatoes using the Pheromone Mixture (3E,8Z,11Z)-tetradecatrien-1-yl-acetate-(3E,8Z)- tetradecadien-1-yl acetate According to the Invention and Outside Invention

Principle: 3 modalities for treating tomatoes in greenhouses are studied in parallel:
Modality 1 (comparative): Normal chemical treatment by the farmer (depends on the country).
Modality 2 (comparative): Mating disruption treatment using Isonet® T³ dosed at 60 g of pheromonel mixture per ha. Analysis of the pheromone mixture indicates a pheromone content of 85% and an impurity level resulting from a substitution in position 3 of 2.4%, which corresponds to a pheromone mixture outside the invention.
Modality 3 (invention): Treatment in mating confusion with the pheromonal mixture of example 5, formulated by microencapsulation according to patent EP3258780A1 and dosed at 46.5 g/ha. The product containing the mixture of active agents is deposited in the form of 1.1 g dots at the rate of 700 dots per ha.

The treatments according to the 3 modalities were implemented in a greenhouse on the same farm, the whole constituting a test. These tests were repeated in different locations in Europe to obtain a statistical picture of the comparative performance of the 3 treatment modalities. Each test was carried out over a period of 3 months and the tests took place between November 2019 and November 2020. The effectiveness of the mating disruption products of modalities 2 and 3 ultimately depends on the pheromonel bouquet perceived by the insects in the ambient air. The effectiveness of this pheromone bouquet depends on the purity and impurity profile of the active ingredients used, as well as the dose emitted. All the tests were carried out over the same duration and it was found at the end of each test that the residual level of active ingredient in the diffusers was zero. The tests according to modality 3 therefore always emitted less pheromone per day than the tests according to modality 2 (46.5 g/ha v. 60 g/ha) and despite this, the superiority of the composition of pheromones according to the invention has demonstrated its superiority compared to market references outside the invention as illustrated by the results below, demonstrating the importance of a low impurity content resulting from a substitution in position 3.

### Tests are Tracked in 3 Different Ways:

A) Weekly trapping records: for each treatment modality, between 3 and 5 pheromone traps are randomly placed and the number of moths trapped is recorded each week. If mating confusion works, the level of trapping must be much lower in modalities 2 and 3 than in modality 1. Trapping has no protective function by mating confusion but only a function of measuring the intensity of presence of Tuta absoluta in cultures.
B) Records at the end of the test (3 months) of damage to stems and leaves: for each modality of treatment, the number of traces of Tuta absoluta in a space of 2 m of row is evaluated at 5 different places in the greenhouse, selected randomly, and the average percentage of leaves or stems damaged by the insect is reported.
C) Records at the end of the test (3 months) of damaged fruits: for each modality of treatment, the number of fruits damaged by the insect is evaluated on 50 fruits randomly sampled from 5 different places in the greenhouse.

The test results are then analyzed to assess whether these results are statistically equivalent. The tests are grouped by type of insect pressure.

Number of tests carried out: 15 (Italy, France, Spain).

Surface of the greenhouses for the tests: between 0.5 ha and 1 ha per treatment modality (each test having equivalent surfaces for each different treatment modality). The results of the statistical analysis of the tests are presented below.

### Cumulative Trapping Per Trap Over 3 Months:

On 7 high pressure tests, the following results are obtained:
Modality 1: average 1256 (max. >1800) moths trapped
Modality 2: 248 (max. 568) moths trapped
Modality 3: 266 (max. 521) moths trapped

These results indicate a strong reduction in trapping for modalities 2 and 3 and validate the principle that treatments according to modalities 2 and 3 reduce the pressure of the Tuta absoluta in an equivalent manner.

On 8 low or medium pressure tests:
Modality 1: 153 (max. 280) moths trapped
Modality 2: average 13.25 (max. 24) moths trapped
Modality 3: 22.5 (max. 43) moths trapped

Similar to the results obtained for high insect pressure, treatments according to modalities 2 and 3 reduce the pressure of Tuta absoluta.

### Percentage of Damage to Leaves and Stems:

| | average percentage | maximum percentage |
|---|---|---|
| Modality 1 | 19.1 | 54.2 |
| Modality 2 | 8.1 | 15.1 |
| Modality 3 | 5.3 | 7.5 |

The results presented above show that the treatment according to modality 3 (invention) allows to reduce the damage to the leaves and stems whether this is compared to an ordinary chemical treatment (modality 1) or by another mating disruption treatment (modality 2).

### Percentage of Fruit Damage:

| | average percentage | maximum percentage |
|---|---|---|
| Modality 1 | 24 | 54.2 |
| Modality 2 | 13.9 | 24.0 |
| Modality 3 | 9.5 | 16.2 |

Treatment according to modality 3 reduces the damage of the Tuta absoluta on the fruits compared to a conventional chemical treatment (Modality 1) or another mating disruption treatment (Modality 3).

### Conclusion:

This example illustrates that the pheromone mixture according to the invention allows to have equivalent or even superior results for the protection of tomatoes in greenhouses compared to the other examples, whereas the dose used is significantly lower (46.5 g/ha against 60 g/ha, that is to say 23% less between modalities 2 and 3). The difference in purity (approximately+10% in favor of the pheromone mixture according to the invention) and the much lower level of impurities resulting from the substitutions in position 3 in the pheromone according to the invention (−45%) largely explain this difference in efficiency.

1. A method for synthesizing a pheromone selected from the group consisting of compound (I), compound (I′), and a mixture of the compounds (I) and (I′): 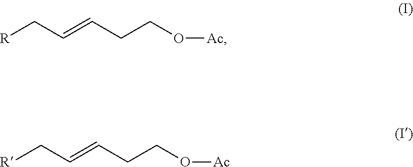
wherein:
R is a linear hydrocarbon chain of molecular formula CₙH₂ₙ₋₂ₚ₊₁,
R′ is a linear hydrocarbon chain of molecular formula C_{n′}H_{2n′-2p′+1},
n and n′, which are identical or different, are integers from 5 to 17
the hydrocarbon chains of molecular formula CₙH₂ₙ₋₂ₚ₊₁ and of molecular formula C_{n′}H_{2n′-2p′+1} each have a number of unsaturations and p and p′ corresponding to the number of unsaturations of the corresponding hydrocarbon chain and are integers, which are identical or different, from 1 to 3, and
Ac is an acetyl group,
the method comprising reacting (2E)-pentene-1,5-diyl diacetate of formula (II): 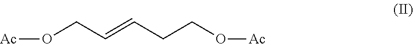 [SMILES: CC(=O)OC/C=C/CCOC(C)=O]
with a Grignard reagent selected from the group consisting of compound (III), compound (III′), and a mixture of compound (III) and (III′):
R-MgX (III), R′-MgX (III′),
wherein R and R′ are as defined previously for the compounds (I) and (I′), and
X is a chlorine or bromine atom;
in the presence of a copper-based catalyst.

2. The method according to claim 1, wherein the copper-based catalyst comprises at least one copper ligand selected from the group consisting of a trialkylphosphine, a trialkylphosphite, an amine, and mixtures thereof.

3. The method according to claim 2, wherein the copper ligand is a trialkylphosphite selected from the group consisting of a triethylphosphite, a trimethylphosphite and mixtures thereof.

4. The method according to claim 1, wherein the reaction is carried out in an aprotic solvent.

5. The method according to claim 1, wherein the copper-based catalyst is a copper II salt.

6. The method according to claim 14, wherein the copper halide is selected from CuCl₂ and CuBr₂; and the copper carboxylate is selected from copper acetate Cu(OAc)₂ and copper II acetyl acetonate Cu(Acac)₂.

7. The method according to claim 1, comprising the following steps:
a. preparing, in an aprotic solvent, a solution comprising 1 equivalent of (2E)-pentene-1,5-diyl diacetate of formula (II);
b. adding to the solution obtained in step a) the copper-based catalyst at the rate of 0.001 to 0.02 equivalent of copper;
c. preparing, in an aprotic solvent identical to or different from the aprotic solvent of step a), a solution comprising from 0.95 to 1.25 equivalents of the Grignard reagent;
d. adding the solution obtained in step c) to the solution obtained in step b) with stirring and at a reaction temperature below 25° C. to obtain a first reaction medium wherein a reaction between the (2E)-pentene-1,5-diyl diacetate of formula (II) and the Grignard reagent is performed;
e. stopping the reaction of step d) in order to obtain a second reaction medium; and
f neutralizing the second reaction medium obtained in step e) and extracting the pheromone.

8. The method according to claim 1, wherein the compounds (III) and (III′) are used in a compound (III): compound (III′) molar ratio and the compound (III): compound (III′) molar ratio is from 0:1 to 1:0.

9. A composition comprising a pheromone selected from the group consisting of compound (I), compound (I′), and a mixture of the compounds (I) and (I′): 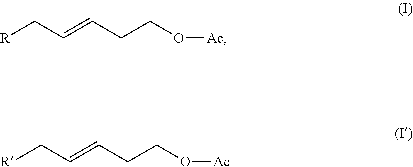
wherein:
R is a linear hydrocarbon chain of molecular formula CₙH₂ₙ₋₂ₚ₊₁,
R′ is a linear hydrocarbon chain of molecular formula C_{n′}H_{2n′-2p′+1},
n and n′, which are identical or different, are integers from 5 to 17;
the hydrocarbon chains of molecular formula CₙH₂ₙ₋₂ₚ₊₁ and of molecular formula C_{n′}H_{2n′-2p′+1} each have a number of unsaturations and p and p′ corresponding to the number of unsaturations in the corresponding hydrocarbon chain and are integers, which are identical or different, from 1 to 3; and
Ac is an acetyl group, and
containing less than 2%, in % by weight of the composition, of a compound selected from the group consisting of compound (VI), compound (VI′), and a mixture of the compounds (VI) and (VI′):

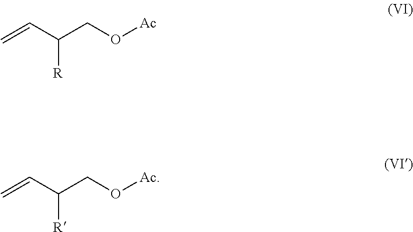

10. (canceled)

11. A method for protecting an agricultural plot or a crop against the Tuta absoluta pest comprising the application within the agricultural plot or the crop, of a composition according to claim 9.

12. The method according to claim 4, wherein the aprotic solvent is an ether type solvent.

13. The method according to claim 4, wherein the aprotic solvent is selected from the group consisting of diethyl ether, methyltertbutyl ether, tetrahydrofuran, methyltetrahydrofuran, tetrahydropyran, methyltetrahydropyran, dioxane, glycol dimethyl ether, and mixtures thereof.

14. The method according to claim 1, wherein the copper-based catalyst is selected from the group consisting of copper II halides and copper II carboxylates.

15. The method according to claim 7, wherein the copper is bound to a ligand selected from the group consisting of a trialkylphosphine, a trialkylphosphite, an amine, and mixtures thereof.

16. The method according to claim 7, wherein stopping the reaction consists of adding 0.1 to 1.5 equivalent of acetic anhydride to the first reaction medium.

17. The composition according to claim 9, wherein the composition contains less than 1.6%, in % by weight of the composition, of the compound selected from the group consisting of compound (VI), compound (VI′), and a mixture of the compounds (VI) and (VI′).
